(19) United States
(12) Patent Application Publication
(10) Pub. No.: US 2013/0310872 A1
(43) Pub. Date: Nov. 21, 2013

(54) PORTABLE PNEUMATIC ABDOMINAL AORTIC COMPRESSION SYSTEM

(71) Applicants: John M. Croushorn, Birmingham, AL (US); Ted Westmoreland, Greenville, SC (US); Richard Schwartz, Birmingham, AL (US)

(72) Inventors: John M. Croushorn, Birmingham, AL (US); Ted Westmoreland, Greenville, SC (US); Richard Schwartz, Birmingham, AL (US)

(21) Appl. No.: 13/983,500

(22) PCT Filed: Feb. 3, 2012
(86) PCT No.: PCT/US2012/023886

§ 371 Date: Aug. 2, 2013

Related U.S. Application Data
(60) Provisional application No. 61/439,628, filed on Feb. 4, 2011.

(51) Int. Cl. A61B 17/135; A61B 17/132

(52) U.S. Cl. CPC A61B 17/135; A61B 17/1325
USPC 606/202

(57) ABSTRACT

A portable pneumatic abdominal aortic tourniquet for occlusion of the abdominal descending aorta to restrict blood supply to a non-compressible arterial hemorrhage in the abdominal region. The tourniquet comprising an adjustable waist strap for securing around an abdomen; a directed air bladder mounted to the waist strap having a generally “V” shaped construction operable between a deflated condition wherein the directed air bladder is collapsed, and an inflated condition wherein the directed air bladder is expanded for exerting pressure against the abdomen; and, an air source connected to the directed air bladder for operating the directed air bladder between the deflated condition and the inflated condition.

### CROSS REFERENCE TO RELATED APPLICATION

This application is a Continuation-In-Part of pending U.S. patent application Ser. No. 12/150,728 filed Apr. 30, 2008 which, in turn, claims priority to expired U.S. Provisional Application Ser. No. 60/915,642 filed May 02, 2007 both of which are incorporated herein by reference. This application claims priority to pending to U.S. Provisional Application Ser. No. 61/439,628 filed Feb. 4, 2011 which is incorporated herein by reference.

### BACKGROUND OF THE INVENTION

The present invention relates to tourniquets, and more particularly, to a portable pneumatically operated aortic tourniquet device that consists of a waistband is and an inflatable air bladder in combination with either a manual pump or a compressed gas injection cartridge for inflating the bladder. The air bladder is of a triangular, cuneiform shape so that a directed compression force is applied to the abdomen specifically to cut-off blood flow to the abdominal descending aorta. A windlass rod provides a tightening mechanism.

The prior art is replete with various tourniquet devices that use a wide variety of clamping and/or pneumatic means to apply pressure to various limbs on the body. However, the prior art does not address the problems associated with abdominal aortic compression in the field in a rapid application portable package for restricting blood flow for a non-compressible arterial hemorrhage in the abdominal region. Such a wound requires occlusion of the abdominal descending aorta to cut-off the blood supply to the non-compressible arterial hemorrhage.

One of the major obstacles to providing an effective portable abdominal aortic tourniquet is in providing a focused compression force over a designated area on the abdomen. Typically, most tourniquets apply a constricting force around the circumference of a limb or over a broad area to reduce total blood flow through the limb. Such a broad application of force is ineffective to reduce or occlude blood flow through the descending aorta proximal to the bifurcation in the abdomen due to the deep location of the aorta in the body. A strong focused pressure is required to reach the descending aorta and reduce blood flow. The prior art fails to show or disclose a pneumatic tourniquet having a directed air bladder able to focus the compression force of the tourniquet sufficient to operate as an abdominal aortic tourniquet.

US Patent Application Publication No. 2007/0191881 A1 (Amisar et al.) shows a tourniquet that includes a pressure source and a selector leaver attached to a cam to facilitate manual selection of a designated pressure. This tourniquet is designed to apply pressure around a limb. There is no teaching in the patent that this device would be effectively useable as an abdominal aortic tourniquet. Further, the air bladder is not a directed air bladder that would focus the compression force, but is rounded to wrap around the limb and spread the pressure force over a broad area. Such a broad application of constricting force is unusable if intended to reduce or occlude circulation through the descending aorta for a non-compressible arterial hemorrhage in the abdominal region.

U.S. Pat. No. 5,234,459 (Lee) shows an inflatable balloon for use in a tourniquet. The patent discloses a manual pump for inflating the balloon. There is no disclosure of the balloon having a directed shape for focusing a compression force, or that the tourniquet is in any way designed to work as an abdominal aortic tourniquet. This tourniquet is representative of a vast majority of pneumatic prior art tourniquet devices which completely fail to address the specific problems associate with providing an effective abdominal aortic tourniquet

U.S. Pat. No. 6,884,254 (Brooks) shows a tourniquet system that includes a leverage assisted clamp means for tightening the strap around a limb. This patent is representative of a large section of the prior art that uses mechanical means, as opposed to pneumatic to provide a constricting force around a limb. Again, such devices fail to provide the directed compression force required to restrict blood flow through the descending aorta.

Accordingly, there is a need for a portable abdominal aortic tourniquet that can be rapidly applied under field conditions.

### SUMMARY OF THE INVENTION

It is an object of the present invention to provide means for applying pressure on the exterior abdomen to reduce or occlude the flow of blood through the descending aorta proximal to the bifurcation.

It is an object of the present invention to provide a pneumatic device with sufficient compressing force to operate effectively as an abdominal aortic tourniquet.

It is an object of the present invention to provide a pneumatic tourniquet having a directed air bladder to focus a compressing force along a defined narrow section of the abdomen to reduce or occlude the flow of blood through the descending aorta proximal to the bifurcation.

It is an object of the present invention to provide an easily portable abdominal aortic pneumatic tourniquet that can be rapidly applied in the field.

The above objectives are accomplished according to the present invention by providing a pneumatic abdominal aortic tourniquet comprising an adjustable waist strap for securing around an abdomen; a rigid base plate carried by the waist strap having a width greater than the waist strap so that the base plate extends laterally outward from the waist strap to provide a stable base for positioning over a selected area of the abdomen; a directed air bladder carried on a bottom side of the base plate has a deflated condition wherein the directed air bladder is collapsed against the base plate, and an inflated condition wherein the directed air bladder is expanded to extend outwardly from the bottom side of the base plate; the directed air bladder having a generally “V” cuneiform shaped construction so that a wide end of the directed air bladder is generally carried on the bottom side of the base plate and a narrow end of the directed air bladder presses against the abdomen when in the inflated condition so that a constricting force caused by inflation of the directed air bladder against the abdomen is focused against a narrow defined area of the abdomen to restrict blood flow through the abdominal aorta; and, an air source operatively connected in fluid communication with the directed air bladder for operating the directed air bladder between the deflated condition and the inflated condition.

In a further embodiment, the tourniquet includes a compression latch carried on the base plate operatively associated with the waist strap for tightening the waist strap around the abdomen and a windlass rod to quickly secure the tourniquet around the body. Preferably, the compression latch includes a latch base mounted to a top side of the base plate in a fixed arrangement with a first distal end of the waist strap; a latch arm pivotally mounted to the latch base operable between a closed position adjacent the latch base and an open position extending upward from the latch base; and, a terminal pivot arm pivotally carried by the latch arm having a second distal end of the waist strap secured thereto, wherein the terminal pivot arm is moved from a relaxed position to a tightened position when the latch arm is operated from the open position to the closed position so that the waist strap is shortened to tighten around the abdomen.

The tourniquet includes an optional foam pad carried on the bottom side of the base plated disposed between the directed air bladder and the base plate to cushion the base plate against the abdomen when the directed air bladder is in the deflated condition. Preferably, a peripheral edge of the foam pad extends beyond a peripheral edge of the base plate on all sides for cushioning against the abdomen before inflation of the directed air bladder.

In a further embodiment, the tourniquet includes a protective bladder sleeve surrounding the directed air bladder in both the inflated and collapsed conditions to resist puncture and protect against environmental exposure of the directed air bladder. Preferably, the protective bladder sleeve is attached to the directed air bladder so that the protective bladder sleeve is collapsed against the directed air bladder when the directed air bladder is in the deflated condition.

In a further embodiment, the tourniquet includes an inflation control to valve carried by the air bladder in fluid communication with the air source and an interior cavity of the directed air bladder for controlling the flow of air into and out of the directed air bladder. Preferably, the inflation control valve is selected from the group consisting of a Presta valve and a Schrader valve. Further, it is preferred that the air source comprises a compressed gas cartridge.

In a further embodiment, the inflation control valve extends through the base plate for cooperating with the air source on a top side of the base plate. Preferably, an elbow connecting valve is disposed between the inflation control valve and the compressed gas cartridge so that the compressed gas cartridge extends generally parallel to the base plate when engaged with the elbow connecting valve.

In a further embodiment, the tourniquet includes a pressure relief valve operatively associated with the directed air bladder for adjusting an air pressure within the directed air bladder when in the inflated condition.

In an alternative embodiment, the air source includes a manual bulb pump having an air supply line connected to the directed air bladder in fluid communication for injecting air into the directed air bladder when the bulb pump is operated. Preferably, a pressure relief valve is carried by the air supply line for adjusting an air pressure within the directed air bladder when in the inflated condition.

In a further embodiment, the tourniquet includes at least one guide marker carried on the base plate for aligning the base plate on the abdomen over the abdominal aorta.

### BRIEF DESCRIPTION OF THE DRAWINGS

The construction designed to carry out the invention will hereinafter be described, together with other features thereof. The invention will be more readily understood from a reading of the following specification and by reference to the accompanying drawings forming a part thereof, wherein an example of the invention is shown and wherein:

FIG. 1 shows a perspective view of a pneumatic abdominal aortic tourniquet attached in an operative condition to a person according to the present invention.

FIG. 1A shows a human form representation indicating where the pneumatic abdominal aortic tourniquet is to be applied according to the present invention.

FIG. 2A shows a perspective view of the pneumatic abdominal aortic tourniquet in a deflated condition being secured around a persons lower abdomen according to the present invention.

FIG. 2B shows a perspective view of the waist strap for the pneumatic abdominal aortic tourniquet being tightened around a persons lower abdomen according to the present invention.

FIG. 2C shows a perspective view of the pneumatic abdominal aortic tourniquet in an inflated condition according to the present invention.

FIG. 3 shows a detailed perspective view of the pneumatic abdominal aortic tourniquet according to the present invention.

FIG. 4A shows a top plan view of the base plate of the pneumatic abdominal aortic tourniquet according to the present invention.

FIG. 4B shows a front elevation view and cut-away of the base plate carrying the inflatable air bladder and protective sleeve covering the air bladder according to the present invention.

FIG. 4C shows a side elevation view of the base plate carrying the inflatable air bladder according to the present invention.

FIG. 5A shows a detailed exploded view of the air source, elbow connector and inflation control valve according to the present invention.

FIG. 5B shows an alternative embodiment of the elbow connector according to the present invention.

FIG. 6 shows an alternative embodiment of the pneumatic abdominal aortic tourniquet having an inflation pump according to the present invention.

FIG. 7 shows an alternative embodiment of the pneumatic abdominal aortic tourniquet according to the present invention.

FIG. 8 shows an alternative embodiment of the pneumatic abdominal aortic tourniquet according to the present invention in partial exploded view.

FIG. 9 shows an alternative embodiment of the pneumatic abdominal aortic tourniquet according to the present invention as in use.

### DETAILED DESCRIPTION OF A PREFERRED EMBODIMENT

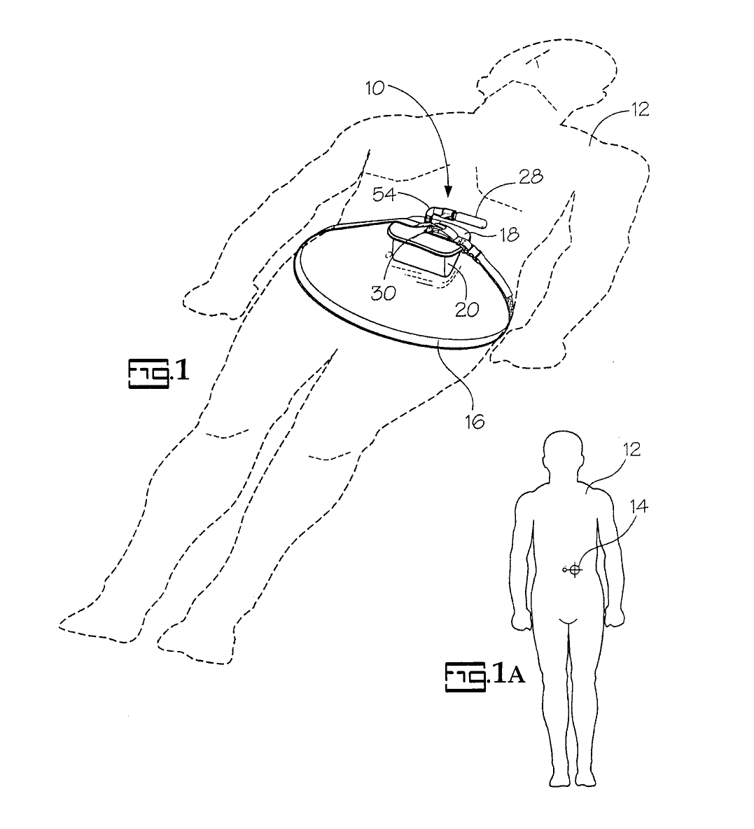

With reference to the drawings wherein similar elements are numbered accordingly, the invention will now be described in more detail. Referring to FIG. 1, a pneumatic abdominal aortic tourniquet, designated generally as 10, is shown secured around the lower abdomen of a person 12 for restricting blood flow through the descending aorta proximal to the bifurcation to deal with a non-compressible arterial hemorrhage in the abdominal region. Referring to FIG. 1 A, the tourniquet is arranged over the abdomen to apply localized pressure across a pressure point 14 to restrict blood flow.

Referring to FIGS. 1 and 3, pneumatic abdominal aortic tourniquet 10 includes an adjustable waist strap 16 for securing around the abdomen. A rigid base plate 18 is carried on waist strap 16. Base plate 18 is constructed and arranged to have a width greater than waist strap 16 so that base plate 18 extends laterally outward from waist strap 16 to provide a stable base for positioning over and across a selected area, such as pressure point 14 (FIG. 1A), of the abdomen.

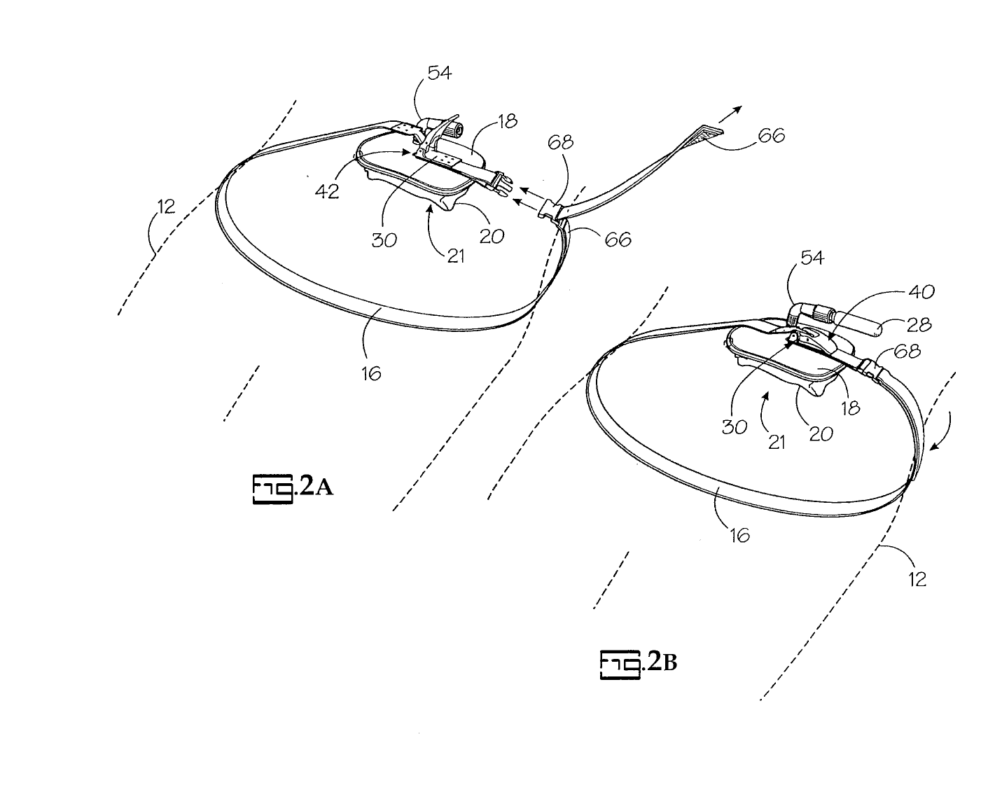

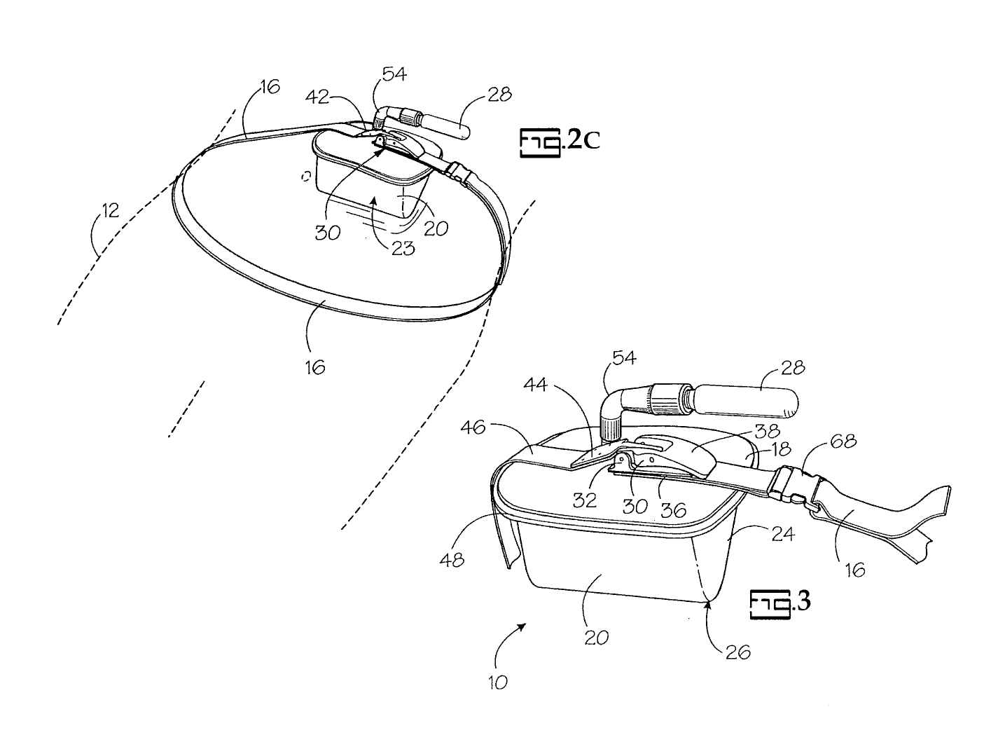

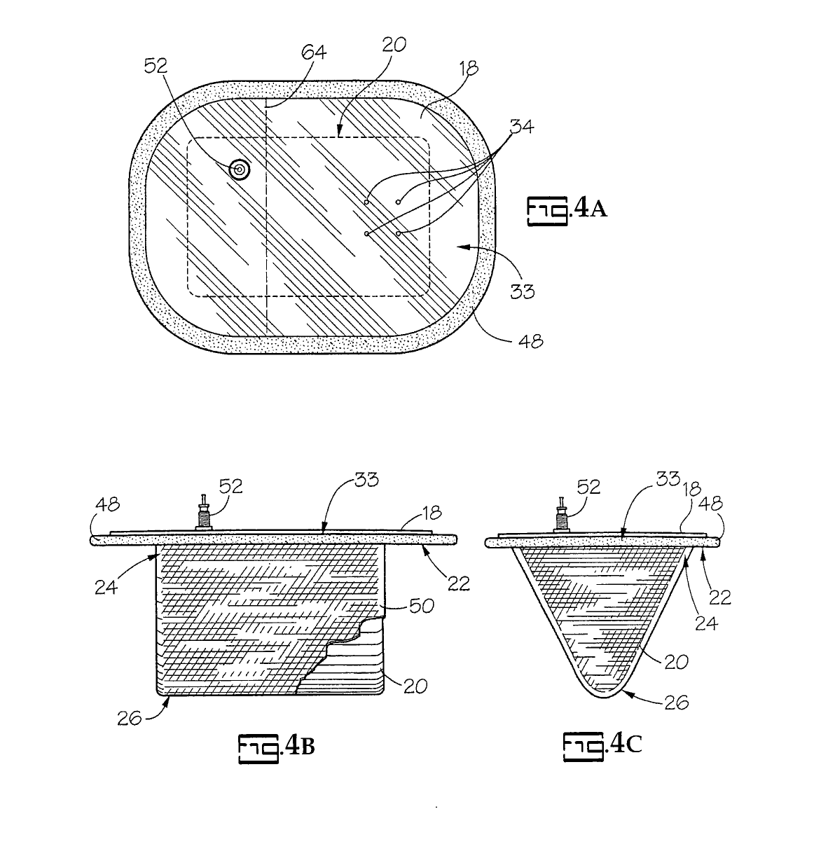

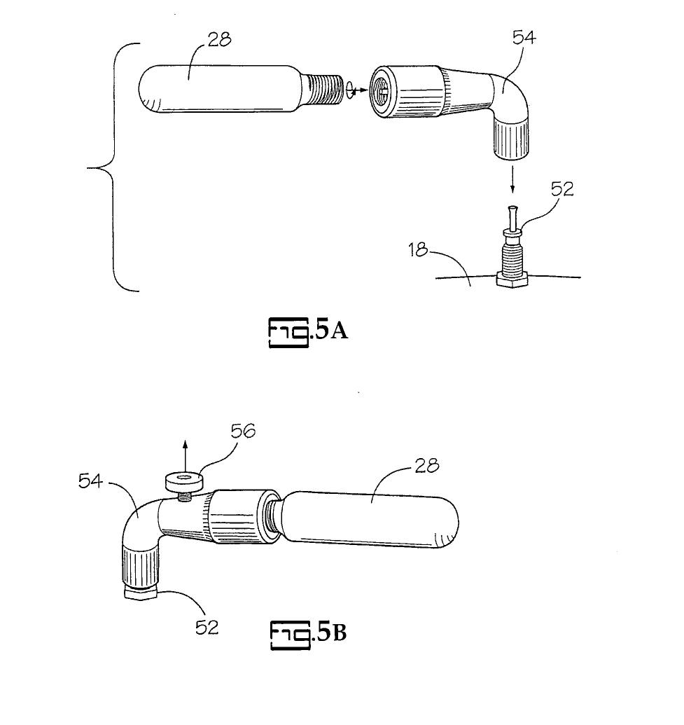

A directed air bladder 20 is carried on a bottom side 22 (FIG. 4B) of base plate 18. As shown in FIGS. 2A and 2B, directed air bladder 20 has a deflated condition, designated generally as 21, for initial installation wherein directed air bladder 20 is generally collapsed against base plate 18. Referring to FIG. 2C, directed air bladder 20 is then operated to an inflated condition, designated generally as 23, wherein directed air bladder 20 is expanded to extend outwardly from bottom side 22 of base plate 18 to exert pressure on a localized area of the abdomen. Referring to FIG. 4C, directed air bladder 20 is constructed and arranged to have a generally “V” shaped construction so that a wide end, designated generally as 24, of directed air bladder 20 is carried on bottom side 22 of base plate 18. A narrow end 26 of directed air bladder 20 presses against the abdomen, generally parallel to the base plate, when in the inflated condition (FIG. 2C) so that a constricting force caused by inflation of directed air bladder 20 against the abdomen is focused against a narrow elongated defined area of the abdomen to restrict blood flow through the abdominal aorta.

Referring to FIG. 3, an air source 28 is operatively connected in fluid communication with directed air bladder 20 for operating the directed air bladder between deflated condition 21 and inflated condition 23. In one embodiment the air source 28 comprises a compressed gas cartridge, such as a CO₂ cartridge well known to those skilled in the art.

Referring to FIG. 3, tourniquet 10 may include a compression latch 30 carried on base plate 18 operatively associated with waist strap 16 for tightening waist strap 16 around the abdomen. Preferably, compression latch 30 includes a latch base 32 mounted to a top side 33 (FIG. 4C) of base plate 18 in a fixed arrangement. Referring to FIG. 4A, openings 34 may be included in base plate 18 for receiving rivets, screws or the like for mounting latch base 32 to top side 33 of base plate 18 in a secure arrangement. Referring to FIG. 3, in a preferred embodiment, a first distal end 36 of waist strap 16 is disposed between latch base 32 and top side 33 of base plate 18 to secure base plate 18 to waist strap 16. A latch arm 38 is pivotally mounted to latch base 32 and is operable between a closed position, designated generally as 40, wherein latch arm 38 is adjacent latch base 32, and an open position, designated generally as 42 (FIG. 2A), extending upward from latch base 32. A terminal pivot arm 44 is pivotally carried by latch arm 38 having a second distal end 46 of waist strap 16 secured thereto. Terminal pivot arm 44 moves from a relaxed position (FIG. 2A) when latch arm 38 is in open position 42, to a tightened position (FIGS. 2B and 2C) when latch arm 38 is moved to closed position 40 so that waist strap 16 is shortened to tighten around the abdomen.

Referring to FIGS. 4A-4C, a foam pad 48 may be carried on bottom side 22 of base plated 18 disposed between directed air bladder 20 and base plate 18 to cushion base plate 18 against the abdomen when the directed air bladder is in deflated condition 21. Preferably, a peripheral edge of foam pad 48 extends beyond a peripheral edge of base plate 18 on all sides for cushioning against the abdomen before inflation of directed air bladder 20. A foam pad which is about 0.5 cm thick and extends beyond the edges of the base plate by 1 cm is suitable for demonstration of the invention.

Referring to FIG. 4B, a protective bladder sleeve 50 may be provided surrounding directed air bladder 20 in both inflated and collapsed conditions 21 and 23 respectively, to resist puncture and protect against environmental exposure of directed air bladder 20. Preferably, protective bladder sleeve 50 is attached to directed air bladder 20, such as by using an adhesive or making protective bladder sleeve 50 form fitting, so that protective bladder sleeve 50 is collapsed against directed air bladder 20 when in deflated condition 21.

Referring to FIGS. 4A-4C, an inflation control valve 52 is carried by directed air bladder 20. Inflation control valve 52 is place in fluid communication with air source 28 and an interior cavity of directed air bladder 20 for controlling the flow of air into and out of directed air bladder 20 for operation between inflated condition 23 and deflated condition 21. Preferably, inflation control valve 52 is a Presta valve or a Schrader valve. In the illustrated embodiment, inflation control valve 52 extends through base plate 18 for cooperating with air source 28 on top side 33 of base plate 18. In a further embodiment, a pressure gauge is operatively associated with directed air bladder 20 for warning if the pressure is dropping in the bladder or maximum pressure has been reached.

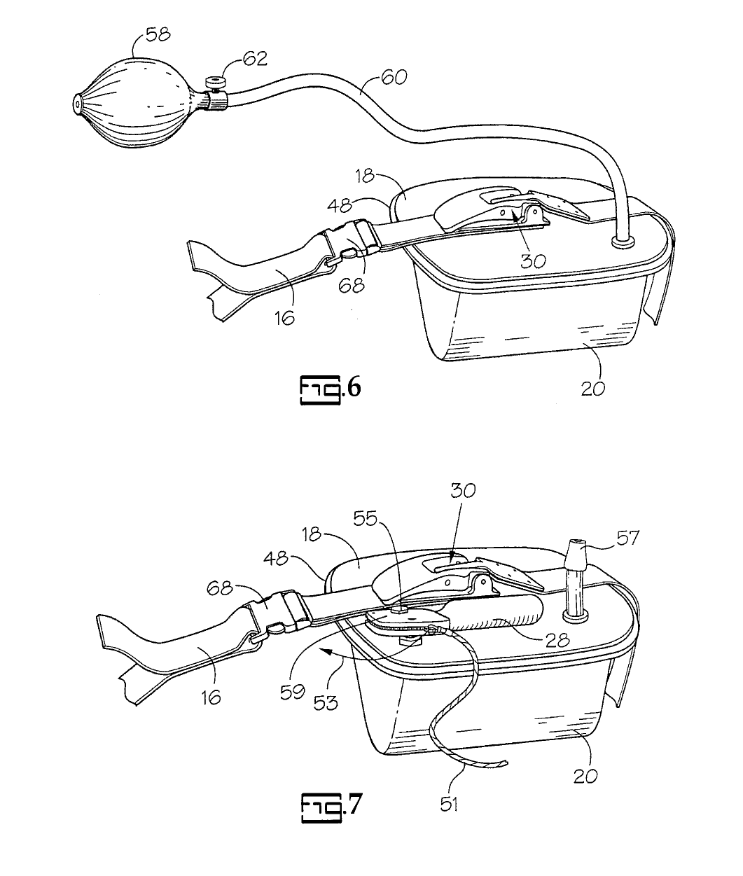

Referring to FIG. 5A, an elbow connecting valve 54 is preferably disposed between inflation control valve 52 and air source 28, in the form of a compressed gas cartridge, extends generally parallel to base plate 18 when engaged with elbow connecting valve 54. This provides a lower profile to the design to help avoid accidental contact with air source 28 that may result in disengagement and deflation of directed air bladder 20.

Referring to FIG. 5B, in a further embodiment, a pressure relief valve 56 is carried by elbow connecting valve 54 which is operatively associated with directed air bladder 20 through inflation control valve 52 for adjusting an air pressure within directed air bladder 20 when in inflated condition 23. Pressure relief valve 56 may alternatively be carried at an alternative location such as directly on air bladder 20, and is not limited to be disposed on elbow connecting valve 54, which is illustrated as the preferred location as a matter of convenient use.

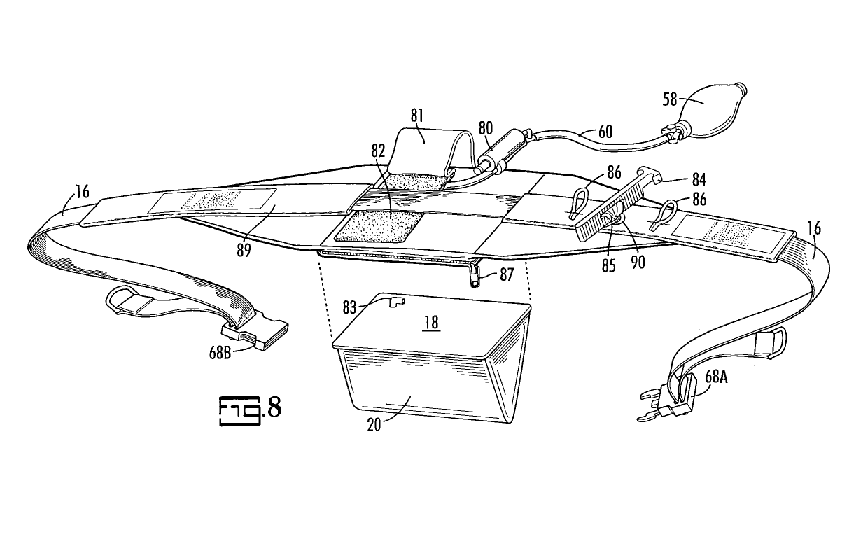

Referring to FIG. 6, in an alternative embodiment, air source 28 includes a manual bulb pump 58 and an air supply line 60 extending from bulb pump 58 to directed air bladder 20 through base plate 18 for injecting air into directed air bladder 20 when bulb pump 58 is operated. Preferably, a pressure relief valve 62 is carried by air supply line 60 for adjusting the air pressure within directed air bladder 20 when in inflated condition 23.

Referring to FIG. 4A, at least one guide marker 64 may be carried on base plate 18 for helping align base plate 18 on the abdomen over and across the abdominal aorta as indicated by pressure point 14 of FIG. 1A.

Preferably, waist strap 16 is constructed of 4 cm wide, 120 cm long, nylon webbing. Referring to FIGS. 2A and 2B, cooperating hook and loop fasteners 66 are preferably provided for securing loose ends of strap 16 onto itself once the strap is drawn tight around the abdomen. Waist strap 16 is of sufficient length to go around the torso just above the iliac crest. Referring to FIG. 3, a quick connect buckle 68 is provided on waist strap 16 for quickly attaching and detaching waist strap 16 around the torso.

Base plate 18 is preferably made of injected molded ridged plastic material. Base plate 18 serves a two fold: first, it is meant to connect the pressure application mechanism, directed air bladder 20, to waist strap 16, and; second, base plate 18 is to provide a stable platform for anchoring directed air bladder 20 on the abdomen to prevent pivotal movement when in inflated condition 23.

Referring to FIGS. 2A-2C, in use waist strap 16 is fed around the patient's body 12 with waist strap 16 lying above the iliac crests. Buckle 68 is then connected to secure waist strap 16 around the torso. Base plate 18 is positioned just left of midline such that the apex of the bladder extends across pressure point 14. The slack is then removed from waist strap 16 and the extra strap 16 is secured onto itself using hook and loop connectors 66. Compression latch 30 is operated from open position 42 to closed position 40 to further tighten waist strap 16. Air source 28 is applied to elbow connector valve 54, typically by screwing a threaded end of a CO₂ cartridge into a complementary threaded receiver on elbow connector valve 54. The cartridge 28 is screwed to its maximum depth to penetrate the cartridge. The cartridge is gently unscrewed slightly to release the compressed gas into directed air bladder 20 through inflation control valve 52. Pressure relief valve 56 may be operated to fine tune the application of force by directed air bladder 20. The flow of CO₂ can be stopped by screwing the cartridge into elbow connector valve 54. A pressure indicator may be incorporated on the device to warn if high pressure exists in the bladder or that the pressure is falling. Generally the bladder is inflated until the desired effect of cessation of bleeding occurs, or the desired effect of preload return to the heart is achieved. If a manual bulb pump is utilized it is operated in the same way as a blood pressure cuff.

An embodiment of the invention is illustrated in FIG. 7. In FIG. 7, the strap 16, base plate 18, air bladder 20, air source 28, compression latch 30, foam pad 48 and quick connect buckle 68, are as described above. A compartment 59 protects the connection between the air source 28 and the bladder 20. The connection is obscured from view in FIG. 7. A closure 53 such as a zipper closes the compartment and a lanyard 51 provides assistance in rapidly accessing the connection. An adaptor 57 provides for an alternative connection such as an auxiliary air pump, an auxiliary pressure relief valve or for manually blowing up the bladder in the case of inoperability.

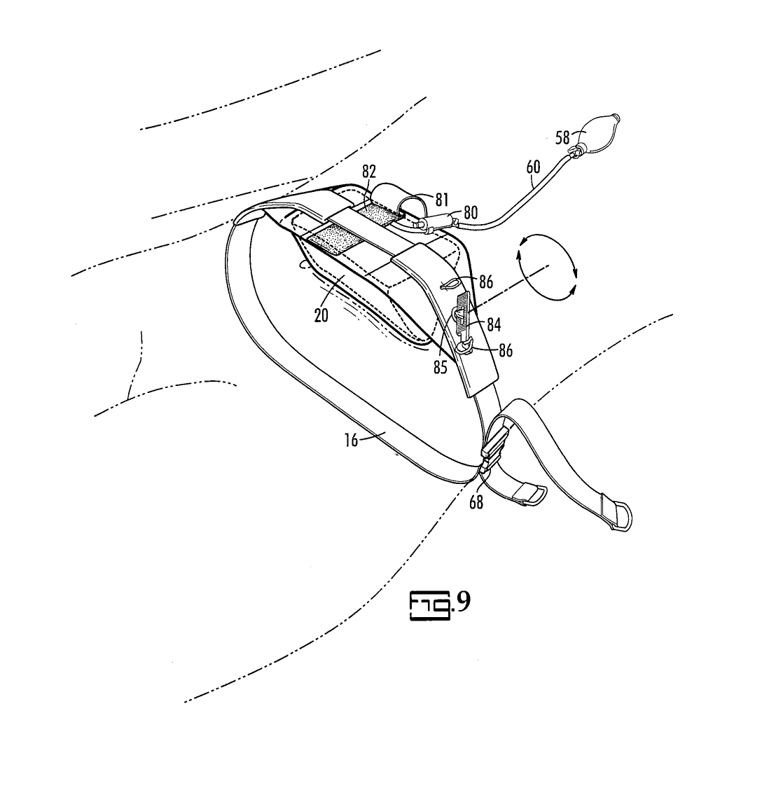

The tourniquet of the present invention is intended for use in trauma situations without limit thereto. In trauma situations timing is of the utmost importance and mere seconds can dictate the ability of the wounded to survive or recover. As a result rapid deployment of the tourniquet is critical. An embodiment of the invention, which will be described with reference to FIGS. 8 and 9, provides a rapid tightening mechanism.

An embodiment of the invention is illustrated in FIGS. 8 and 9. In FIG. 8 the tourniquet is illustrated in partially exploded view and in FIG. 9 the tourniquet is illustrated as in use. A storage device, consisting of a pad 82 and cover 81, provides a storage area for the manual bulb pump 58, the air supply line 60 and a pressure relief valve 80 can be gathered in serpentine fashion, laid on the pad and covered with the cover which preferable forms a closure. The closure can be secured by a hook and latch system such as Velcro®. In one embodiment the tourniquet is intended for one time use and the cover can be irreversibly removed from the pad for access to the contents of the storage area formed by the pad and cover. A connector 83 connects the air bladder to the manual bulb pump and allows the bladder to be replaced in the case of failure. A windlass rod, 84, connected to a portion of the strap 85 allows rapid tightening of the device. Once the tourniquet is applied and the bladder expanded it is difficult to tighten the strap which could normally be done by increasing the length of strap beyond the buckle. It is also not desirable to remove or loosen the tourniquet once it is in place. The windlass rod 84 can be rotated thereby drawing the strap tighter which places more pressure on the abdomen. When the appropriate pressure is achieved the windlass rod can be inserted into a retention loop 86 thereby securing the windlass rod. The windlass rod has a center opening that the strap is routed through. The strap routes up through a grommet 90 in the center of the front shroud 89 of the device and then through the windlass rod and then back through the grommet. The windlass rod is turned either clockwise or counter clockwise until the device is firmly tightened around the waist of the patient. The rod is then secured by a retention loop that is fixed to the front of the device. In one embodiment the strap may be segmented with a segment sewn on one end of the shroud and the other end secured to the windlass rod by either a snap or buckle.

The device may comprise a three piece shroud to aid in routing of the waist strap/belt. It also allows for labeling and functionality of the windlass rod. In one embodiment the shroud is made from 1.2 mil thick reinforced plastic weave sheeting that is water gel cut to form. The three pieces are made of up of a center shroud covering the front of the device and two side pieces that cover the wings of the device. The front center shroud provides for labeling, windlass grommet windlass retention strap and elbow connector from the bladder to the inflation system. The shroud is sewn onto the device. Portions of the perimeter of the shroud are not sewn to create a channel for the waist strap/belt to be routed.

While a preferred embodiment of the invention has been described using specific terms, such description is for illustrative purposes only, and it is to be understood that changes and variations may be made without departing from the spirit or scope of the following claims.

What is claimed is:

1. A pneumatic abdominal aortic tourniquet comprising:
an adjustable waist strap for securing around an abdonmen;
a rigid base plate carried by said waist strap having a width greater than said waist strap so that said base plate extends laterally outward from said waist strap to provide a stable base for positioning over a selected area of said abdomen;
a directed air bladder carried on a bottom side of said base plate having a deflated condition wherein said directed air bladder is collapsed against to said base plate, and an inflated condition wherein said directed air bladder is expanded to extend outwardly from said bottom side of said base plate;
said directed air bladder having a cuneiform shaped construction so that a wide end of said directed air bladder is generally carried on said bottom side of said base plate and a narrow end of said directed air bladder presses against the abdomen when in said inflated condition so that a constricting force by said narrow end caused by inflation of said directed air bladder against said abdomen is focused against a narrow defined area of said abdomen to restrict blood flow through the abdominal aorta under said bladder;
an air source operatively connected in fluid communication with said directed air bladder for operating said directed air bladder between said deflated condition and said inflated condition;
a windlass rod attached to said waist strap wherein rotation of said windlass rod tightens said waist strap; and
a retention loop for retaining said windlass rod in position.

2. The tourniquet of claim 1 including a protective bladder sleeve surrounding said directed air bladder in both said inflated and collapsed conditions to resist puncture and protect against environmental exposure of said directed air bladder.

3. The tourniquet of claim 2 wherein said protective bladder sleeve is attached to said directed air bladder so that said protective bladder sleeve is collapsed against said directed air bladder when said directed air bladder is in said deflated condition.

4. The tourniquet of claim 1 including an inflation control valve carried by said air bladder in fluid communication with said air source and an interior cavity of said directed air bladder for controlling the flow of air into and out of said directed air bladder.

5. The tourniquet of claim 4 wherein said inflation control valve is selected from the group consisting of a Presta valve and a Schrader valve.

6. The tourniquet of claim 1 wherein said air source includes a manual bulb pump having an air supply line connected to said directed air bladder in fluid communication for injecting air into said directed air bladder when said bulb pump is operated.

7. The tourniquet of claim 6 including a pressure relief valve carried by said air supply line for adjusting an air pressure within said directed air bladder when in said inflated condition.

8. The tourniquet of claim 1 including at least one guide marker carried on said base plate for aligning said base plate on said abdomen over the abdominal aorta.

9. The tourniquet of claim 1 further comprising a shroud wherein said strap is received by said shroud.

10. The tourniquet of claim 9 wherein said shroud comprises a grommet and said waist strap extends through said grommet for attachment to said windlass.
